(19) United States
(12) Patent Application Publication
(10) Pub. No.: US 2006/0074059 A1
(43) Pub. Date: Apr. 6, 2006

(54) Isomorphic crystalline habits of 3alpha-hydroxy-21-(1'-imidazolyl)-3beta-methoxymethyl-5alpha-pregnane-20-one

(76) Inventors: Philip A. Goliber, Brookfield, CT (US); Pauline E. Leary, Stanfordville, NY (US); Helen Danagher, Cambridge (GB); Matthew Hartenstein, Glenmoore, PA (US)

Correspondence Address: PURDUE PHARMA, LP, 201 TRESSER BOULEVARD, ONE STAMFORD FORUM, STAMFORD, CT, 06901, US

(21) Appl. No.: 11/211,784

(22) Filed: Aug. 25, 2005

Related U.S. Application Data
(60) Provisional application No. 60/604,447, filed on Aug. 26, 2004.

(51) Int. Cl. A61K 31/58; C07J 43/00

(52) U.S. Cl. 514/176; 540/107

(57) ABSTRACT

The present invention provides stable particles of 3a-hydroxy-21-(1′-imidazolyl)-3β-methoxymethyl-5a-pregnan-20-one (Compound I), which possess and retain a shape and size appropriate for handling and manufacture of large-scale pharmaceutical preparations, even in the absence of further milling. Further provided is a method for obtaining such reproducible, stable particles by subjecting crude Compound I to controlled crystallization conditions comprising slow cooling of a solution of Compound I. Further provided is a pharmaceutical composition of unmilled crystalline Compound I, which does not require milling prior to formulation, and a method of modulating brain excitability using the same.

### CROSS-REFERENCE TO RELATED APPLICATIONS

This application claims priority from U.S. Provisional Application Ser. No. 60/604,447 filed Aug. 26, 2004, which is incorporated herein by reference.

### BACKGROUND OF THE INVENTION

1. Field of the Invention

The present invention relates to isomorphic crystalline habits of the neuroactive steroid 3a-hydroxy-2 1-(1′-imidazolyl)-3β-methoxymethyl-5α-pregnan-20-one having improved properties over previously known crystalline material.

2. Related Art

Uniformity of size and shape of pharmaceutical compounds in particulate form, and the uniformity and stability of the crystalline structure of organic pharmaceutical compounds, impart greater predictability and more consistent bioavailability and pharmacodynamics.

A polymorph can be described as a different crystalline form having a different unit cell structure of a given compound, which may arise due to the packing of molecules within the crystal structure, or by differences in the orientation of molecules, including solvated and hydrated crystal forms in which the packing of molecules includes packing with solvent or water, respectively. The resulting crystalline materials having different polymorphic forms may have distinct physical properties, such as melting point, solubility and x-ray diffraction patterns, even though these compounds are otherwise chemically identical.

It is well established that the polymorphic state of a solid pharmaceutical substance can modify physicochemical properties and stability of drugs. However, not much attention has been paid to different crystal habits of isomorphic forms. A crystal's “habit” refers to the external character (e.g., shape) of the crystal. “Isomorphic forms” refer to crystalline solids having a common unit cell structure. A change in the external shape of a growing crystal without any change in its internal structure results in a different habit. Variation in only the crystal habit may serve to improve certain substance properties. An early-phase pre-formulation program can be undertaken for any pharmaceutical candidate to determine the optimal crystal habit (if any) by analyzing, for example, powder flow characteristics, dissolution, and tableting characteristics, so that the biopharmaceutical and manufacturing properties can be optimized.

U.S. Pat. No. 6,277,838 B1, incorporated herein by reference in its entirety, describes the use of 3α-hydroxylated steroid derivatives for modulating brain excitability in a manner that alleviates stress, anxiety, insomnia, mood disorders (such as depression) and seizure activity. Among these compounds, 3α-hydroxy-21-(1′-imidazolyl)-3β-methoxymethyl-5α-pregnan-20-one (“compound I”) has emerged as a potential anxiolytic and sedative-hypnotic drug. See U.S. Patent Application Publication No. US 2004/0034002, incorporated herein by reference in its entirety; Vanover, K.E. et al., J. Pharmacol. Exp. Ther., 291(3):1317-1323 (1999); and Vanover, K. E. et al., Psychopharmacology, 155:285-291 (2001).

U.S. Patent Application Publication No. US 2004/0034002, describes the preparation of crystalline compound I. Compound I prepared according to these methods may not be optimized for large-scale commercial milling techniques. For example, ball milling may induce a change in the crystallinity of compound I, and may be stressful enough to change crystalline compound I into amorphous compound I. The creation of an amorphous material is often associated with agglomeration and increased chemical reactivity. Such an amorphous material may not be sufficiently stable or sufficiently amenable to use in a large-scale pharmaceutical preparation.

Accordingly, there is a need for preparing a crystalline form of compound I having improved properties.

### SUMMARY OF THE INVENTION

The present invention provides reproducible, stable particles of compound I suitable for use in the manufacture of pharmaceutical dosage forms. The stable particles of compound I of the present invention possess and retain a shape and size appropriate for handling and manufacture of large-scale pharmaceutical preparations, even without subsequent milling.

The present invention further provides a method for obtaining such reproducible, stable particles of compound I. The method involves subjecting compound I to controlled recrystallization conditions. More particularly, the present invention provides a method of recrystallizing compound I, comprising slowly cooling a solution of compound I from an appropriate solvent system.

The present invention further provides a pharmaceutical composition of unmilled crystalline compound I, which does not require milling prior to formulation into a usable pharmaceutical dosage form.

The present invention further provides a method of modulating brain excitability by administering to a subject in need thereof an effective amount of unmilled, crystalline compound I prepared according to the recrystallization methods described herein.

Further embodiments, features, and advantages of the present invention, as well as the structure and operation of the various embodiments of the present invention, are described in detail below with reference to the accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying drawings, which are incorporated herein and form a part of the specification, illustrate the present invention and, together with the description, further serve to explain the principles of the invention and to enable a person skilled in the pertinent art to make and use the invention.

FIG. 1. X-ray powder diffraction (XRPD) scans for compound I recrystallized by rapid, cold recrystallization from acetone (gray line) and for unrecrystallized compound I (blue line).

FIG. 2. XRPD scans for compound I recrystallized by room-temperature evaporation under vacuum from acetonitrile (green and red lines) and for unrecrystallized compound I (blue line).

FIG. 3. XRPD scans for compound I recrystallized by slow, cold recrystallization from isopropanol (black line) and for unrecrystallized compound I (blue line).

FIG. 4. XRPD scans for compound I recrystallized by slow, cold recrystallization from isopropanol before (blue line) and after three-months at ambient conditions (red line).

FIG. 5. Infrared spectrum (IR) for compound I recrystallized by slow, cold recrystallization from isopropanol (red line) and for unrecrystallized compound I (blue line).

FIG. 6. XRPD scans for compound I recrystallized by rapid, cold recrystallization from ethanol (orange line) and for unrecrystallized compound I (blue line).

FIG. 7. XRPD scans for compound I recrystallized by rapid, cold recrystallization from ethanol (purple line) before and after four-months at ambient conditions (orange line).

FIG. 8. XRPD scans for compound I recrystallized by rapid, cold recrystallization from methanol (red line) and for unrecrystallized compound I (blue line).

FIG. 9. Differential Scanning Calorimetry (DSC) temperature scan for compound I recrystallized by rapid, cold recrystallization from methanol.

FIG. 10. Scanning electron micrograph of compound I crystal resulting primarily from “fast” recrystallization.

FIG. 11. Scanning electron micrograph of compound I crystal resulting primarily from “slow” recrystallization.

FIG. 12. Scanning electron micrograph of compound I crystals, recrystallized using hot isopropyl ether with “fast” recrystallization.

### DETAILED DESCRIPTION OF THE INVENTION

Compound I is a crystalline powder with a melting point of approximately 191-197° C. The chemical structure of compound I is shown below and its molecular weight and formula are 428.62 and C₂₆H₄₀N₂O₃, respectively. 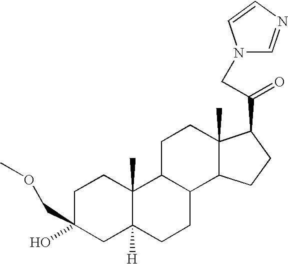
Recrystallization

Samples of compound I (prepared according to the method described in Example 1 of U.S. Patent Application Publication No. US 2004/0034002, incorporated herein by reference in its entirety) were dissolved in test solvents at room temperature. The test solvents included acetone, acetonitrile, isopropanol, ethanol, and methanol. Each dissolved test sample was then divided into four equal-volume aliquots and recrystallized using one of four methods described below. The resulting crystals were characterized.

The final yield for the recrystallized compound I solid samples from some solvents was not enough for characterization. For these samples, a new preparation for each solvent w as heated to a temperature slightly b elow the solvent boiling point and saturated with compound I at this elevated temperature. Each of these test samples was then divided into four equal-volume aliquots and recrystallized using one of four methods described below.

The following four recrystallization methods were employed:
(1) Room Temperature Evaporation Under Vacuum. Sample solutions of compound I were transferred to an oven maintained at room temperature, and dried under vacuum at 30 inches of mercury for up to twenty-four hours.
(2) Elevated Temperature Evaporation Under Vacuum. Sample solutions of compound I were transferred to an oven maintained at approximately 50° C., and dried under vacuum at 30 inches of mercury for up to twenty-four hours.
(3) Slow. Cold Recrystallization from S olvent. Sample solutions of compound I were transferred to a chiller bath maintained at approximately 50° C. The bath was set to cool at a rate of 1° C. per hour to a final temperature of −30° C. When the temperature of the bath reached −30° C. and enough solids had precipitated from the solution for characterization, each solution was decanted from the precipitate and the remaining solids were dried under a stream of nitrogen gas. Note: For solutions where the solvent boiling point was lower than 50° C., the sample solutions were not transferred to the chiller bath until the temperature of the bath was a few degrees lower than the boiling point of the solvent.
(4) Rapid, Cold Recrystallization from Solvent. Sample solutions of compound I, immediately upon reaching saturation, were transferred to a dry ice/acetone slurry. These solutions were maintained under these c onditions for approximately one hour, and then transferred to a chiller bath maintained at −30° C. Sample solutions were maintained at −30° C. overnight or until enough solids had precipitated from the solution for characterization. Each solution was decanted from the precipitate and the remaining solids were dried under a stream of nitrogen gas.

Characterization of Recrystallized Samples

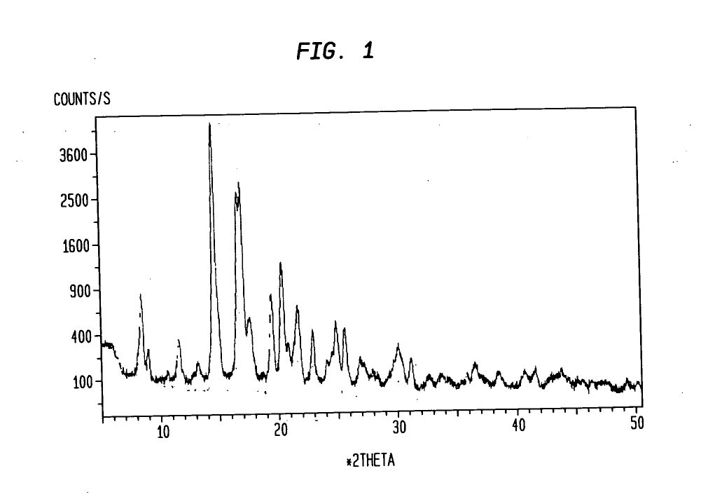

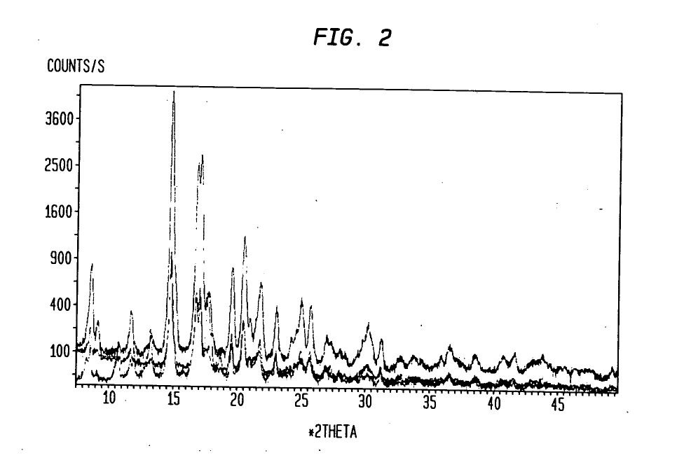

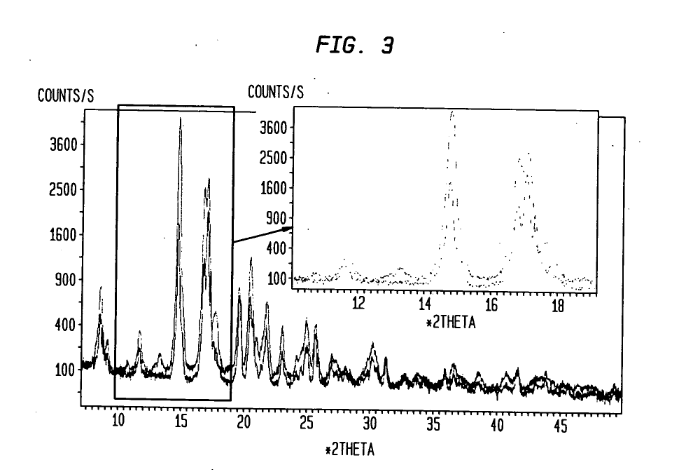

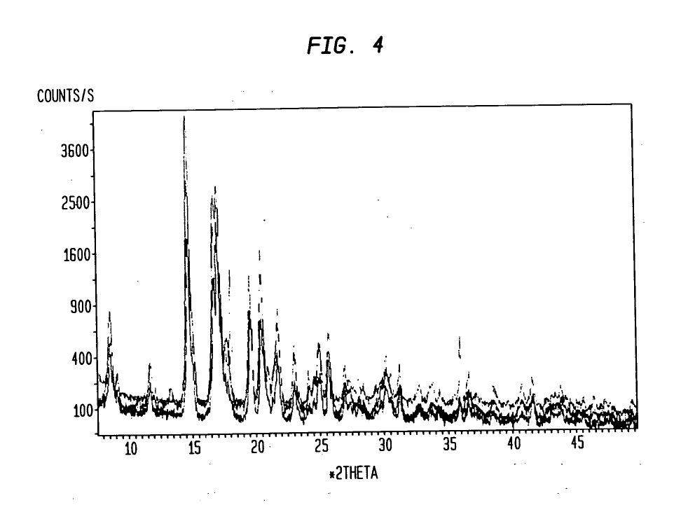

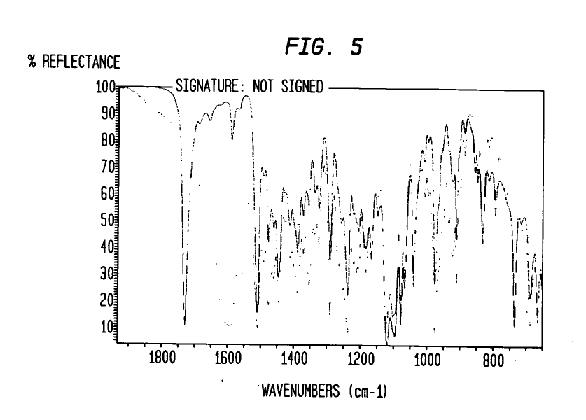

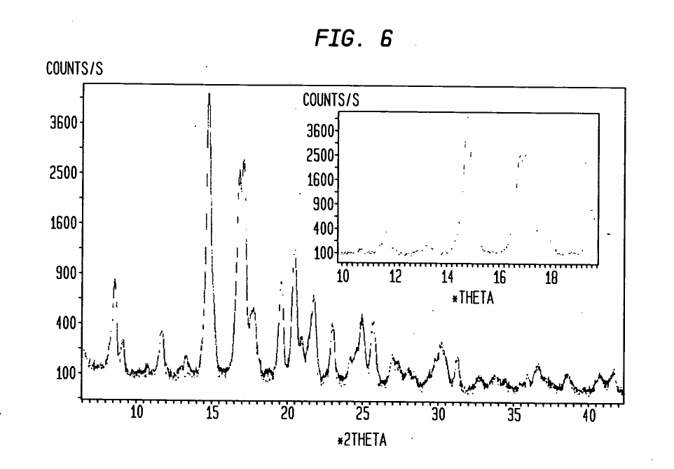

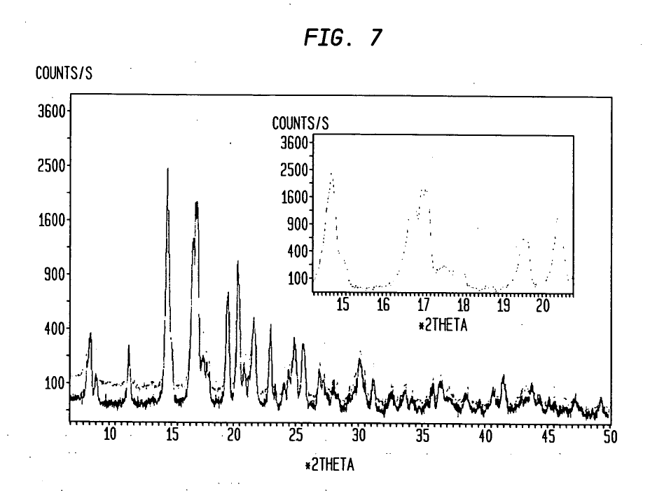

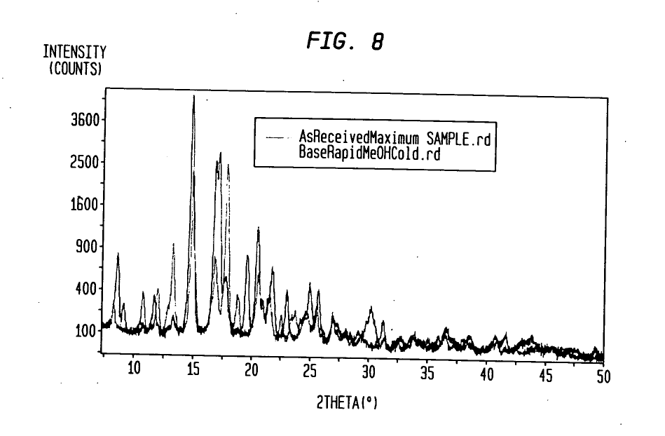

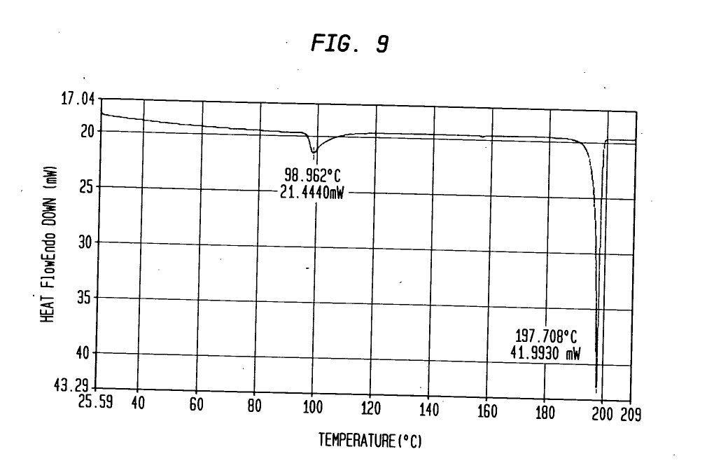

Five different X-ray powder diffraction (XRPD) patterns were identified from samples recrystallized under the controlled conditions described above.
(1) Rapid, cold recrystallization from acetone. The samples of compound I recrystallized from acetone using rapid, cold recrystallization had an XRPD pattern most consistent with the original (i.e., unrecrystallized) sample. A comparison is shown in FIG. 1. The major difference was that the XRPD pattern of the recrystallized samples (gray line) was better resolved, indicating a higher degree of crystallinity. In addition, the reflection in the 2θ range of 17.4-18.4° is a triplet in the recrystallized sample, but is only a singlet in the original sample (blue line). To determine whether the structure of this recrystallized sample was stable over time, the sample was maintained at ambient conditions and analyzed after three months. No remarkable changes were noted after three months, indicating the sample was stable over time.
(2) Room-temperature evaporation under vacuum from acetonitrile. The sample of compound I recrystallized from acetonitrile at room temperature under vacuum had an XRPD pattern different from that of the original (unrecrystallized) sample. A comparison is shown in FIG. 2. In the recrystallized sample (green and red lines), there are peaks at 2θ of 10.7° and 13.2° which, although also present in the original sample (blue line), are of much greater intensity and resolution with narrower, sharper peaks than in the original sample. There is also a difference between these two samples in the 2θ range between 17.3° and 19°. In the recrystallized sample, there is a single peak with a shoulder at the higher 2θ value and relatively high intensity, whereas in the original sample this peak is a s ingle peak with no shoulder. After five months at ambient conditions, no major changes were noted in the XRPD pattern, although small changes were observed including the disappearance of shoulder peaks at 2θ of 11.6° and 12.9°.
In addition to analysis using XRPD, the recrystallized sample was further characterized using Differential Scanning Calorimetry (DSC) and Thermogravimetric Analysis (TGA). The DSC scan exhibited a series of endothermic transitions between 48° C. and 80° C. which can be attributed to thermal events occurring as a consequence of solvent loss. The DSC scan also exhibited a melt endotherm with a peak minimum at 196.109° C. The TGA scan exhibited a 2.2% weight loss in the temperature range between 49° C. and 102° C., also attributed to solvent loss. The XRPD pattern for the recrystallized sample heated to 100° C. for seven minutes was comparable to the original (unrecrystallized) sample, although the peaks in the recrystallized, and heated sample were better defined. The DSC and TGA scans for this sample did not exhibit thermal transitions or weight loss. From the characterization above, it was concluded that compound I sample recrystallized from acetonitrile at room temperature under vacuum is an acetonitrile solvate of compound I.
(3) Slow, cold recrystallization from isopropanol. The sample recrystallized from isopropanol using slow, cold recrystallization had an XRPD pattern different from that of the original (unrecrystallized) sample. A comparison is shown in FIG. 3. Overall, the XRPD pattern of the recrystallized sample (black line) contains peaks that are narrower and sharper than those of the original sample (blue line), indicating a more ordered crystalline structure for the recrystallized sample. Two peaks at approximately 2θ of 10.7° and 13.3° in the XRPD pattern of the original (unrecrystallized) sample are not present in that of the recrystallized sample. In the 2θ range between 16.2° and 17.5°, there is a doublet in the XRPD pattern of the original (unrecrystallized) sample, whereas there is a triplet in that of the recrystallized sample (see expanded region in FIG. 3). In addition, the peak in the 2θ range between 17.4° and 18.4° is a poorly resolved triplet in the XRPD pattern of the recrystallized sample but is a singlet in that of the o riginal (unrecrystallized) s ample ( see e xpanded region in FIG. 3). Finally, the doublet in the 2θ range between 20° and 20.8° in the XRPD pattern of the recrystallized sample is a singlet in that of the original (unrecrystallized) sample.
After three months at ambient c ondition, changes were noted in the XRPD pattern of the recrystallized sample. A comparison is shown in FIG. 4. For instance, after three months (red line) the higher 2θ value shoulder to the peak in the 2θ range between 14° and 15.5° became better resolved; the triplet in the 2θ range between 16.2° and 17.5° converted to a doublet with the same profile as that of the original (unrecrystallized) sample (blue line); an intense, sharp, narrow peak grew in the 2θ range between 17.8° and 18.3°; the doublet in the 2θ range between 10° and 20.8° became a singlet; and the peak in the 2θ range between 35.6° and 36.2° became a more intense doublet.
The infrared spectrum (IR) of the recrystallized sample was different from that of the-original (unrecrystallized) sample (blue line). A comparison is shown in FIG. 5. A strong absorption band between 1690 and 1536 cm⁻¹ is present in the recrystallized sample (red line), indicating that a change in crystal form occurred during the recrystallization.
Based on the characterization above, it was concluded that the sample prepared by slow, cold recrystallization from isopropanol is a meta-stable form of compound I, which converts to a more stable form over time.
(4) Rapid cold recrystallization from ethanol. The sample recrystallized from ethanol using rapid, cold recrystallization had a XRPD pattern different from that of the original (unrecrystallized) sample. A comparison is shown in FIG. 6. There is the emergence of a peak at 2θ of 18.07° in the XRPD pattern of the recrystallized sample (orange line) which is not evident in that of the original (unrecrystallized) sample (blue line).
Differences are noted in the XRPD pattern between the recrystallized sample prior to storage and the recrystallized sample after four months at ambient conditions. These differences are depicted in FIG. 7. The major difference is a split in the peak for the 2θ range between 16.9° and 17.4° (see expanded region in FIG. 7) in the XRPD pattern of the recrystallized sample after four months (orange line).
Based on this information, it was concluded that this sample, like the sample prepared by cold, rapid recrystallization from acetone, has a higher degree of crystallinity than the original (unrecrystallized) compound I of the same polymorphic form.
(5) Rapid, cold recrystallization from methanol. The sample recrystallized from methanol using rapid, cold recrystallization had an XRPD pattern different from that of the original (unrecrystallized) sample. A comparison is shown in FIG. 8 (recrystallized sample in red; original sample in blue). Differences in peak positions and intensities are evident throughout the entire diffraction pattern. The sample was maintained at ambient conditions and analyzed after three months. Although the general characteristics o f t he XRPD patterns were the s ame a fter three months, the three-month XRPD pattern appeared to have gained features consistent with the XRPD pattern of the original (unrecrystallized) sample.
An endothermic transition in the DSC scan for this sample (FIG. 9) at approximately 99° C. is consistent with the postulate that the recrystallized sample is a methanol solvate of compound I.
   Evaluation of Recrystallization Parameters on Crystal Habit

Experiments were performed to evaluate the effect of recrystallization rate, temperature and final drying conditions on the size, shape and crystalline properties of compound I. The conditions and choice of solvent described in the examples below may be varied as determined by those skilled in the art. Thus, the methanol/acetone/isopropyl ether solvent system employed in the examples may be substituted by one or more other appropriate solvent systems as can be determined by those skilled in the art. Appropriate solvent systems include those wherein compound I is: (1) first dissolved in a solvent or mixture of solvents selected from alkanols (R¹OH), chlorinated hydrocarbons, esters (R¹C(O)OR²), ketones (R¹C(O)R²) and the mixtures thereof, wherein R¹ and R² are independently C₁-C₆ alkyl; and (2) then allowed to recrystallize by the slow or fast addition of a co-solvent selected from alkanes (CₙH₂ₙ₊₂), ethers (R³OR⁴), glycols and mixtures thereof, wherein n=5-12; and R³ and R⁴ are independently C₁-C₆ alkyl. The representative solvents include methanol, ethanol, isopropyl alcohol, dichloromethane, chloroform, ethyl acetate, acetone and mixtures thereof, and the representative co-solvents include pentane, hexane, heptane, ethyl ether, isopropyl ether, ethylene glycol, propylene glycol and mixtures thereof. The cooling rate for “slow recrystallization” described in the examples below may also be modified and can include a rate of between about 1° C. per hour to about 10° C. per hour.

(1) Effect of Cooling Rate on Crystal Habit of Compound I. In order to evaluate the impact of cooling rate upon particle shape and size, isopropyl ether was added to a solution of methanol, acetone and crude compound I to induce r ecrystallization. A first s et o f samples w as “fast recrystallized” by transfer of the solution to a dry ice/acetone slurry at about −78° C., followed by transfer to a chiller bath maintained at −30° C. A second set of samples was “slow recrystallized” by cooling at a controlled rate of 5° C. per hour to 0° C.

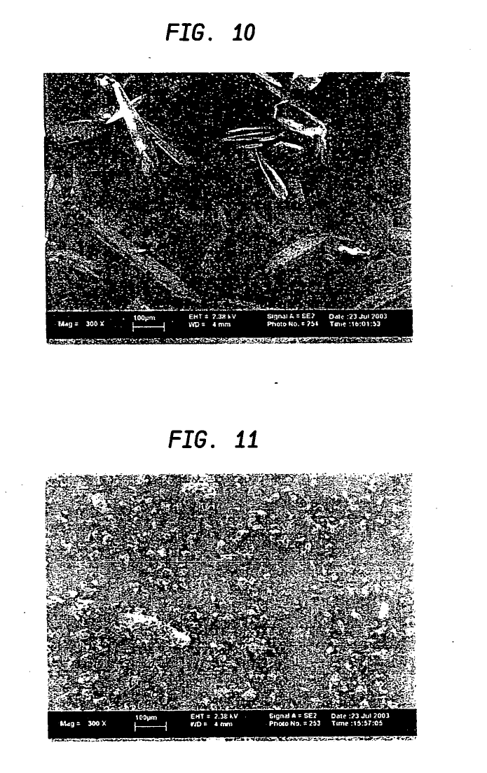

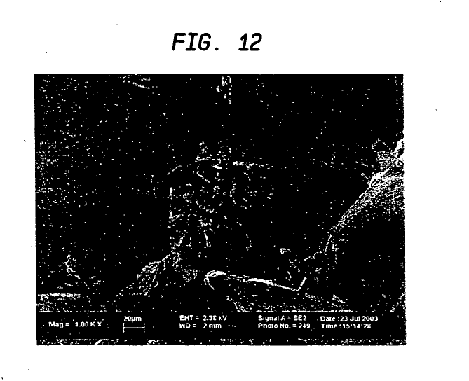

The cooling rate of recrystallization has a substantial impact upon particle shape. The two different cooling rates created two different habits of compound I crystals. Fast recrystallization resulted in primarily elongated plates with tapered edges (scanning electron micrograph displayed in FIG. 10), whereas slow recrystallization resulted in smaller, primarily more shapeless crystalline particles (scanning electron micrograph displayed in FIG. 11). A particular embodiment of the invention comprises crystals as displayed in FIG. 11, having an average crystal size of less than about 100 μm, preferably less than about 50 μm, and more preferably less than about 25 μm.

Importantly, the compound I crystals prepared by slow recrystallization are of a size and shape which makes them amenable to large-scale pharmaceutical formulation processes, even in the absence of further milling.

(2) Effect of Isopropyl Ether Temperature on Crystal Habit of Compound I. In order to evaluate the impact of isopropyl ether (IPE) temperature upon particle shape and size, two different IPE temperatures were evaluated. For a first set of samples, room temperature (about 22 to 25° C.) IPE was added via syringe to a solution of methanol, acetone and crude compound I at reflux (˜65° C.). For a second set of samples, boiling-temperature (˜69° C.) IPE (“hot IPE”) was added to a solution of methanol, acetone and crude compound I at reflux. The samples were then subjected to either “fast recrystallization” or “slow recrystallization” as described above.

The IPE temperature did not affect particle shape appreciably, but did affect crystal size. This effect was more evident in the “fast recrystallized” samples. When hot IPE was added to induce crystallization in “fast recrystallized” samples (i.e., “hot fast IPE”), there were a large number of smaller crystals clustered together in “bursts” as shown by scanning electron microscopy in FIG. 12. When room-temperature IPE was added to induce crystallization in “slow recrystallized” samples, the number of smaller crystals was much less than in the “hot fast IPE” samples, and were not clustered in bursts.

(3) Effect of Drying Conditions on Crystal Habit of Compound I. Three different drying conditions were evaluated. The first set of samples was dried under a stream of nitrogen gas for about twenty hours at room temperature. A second set of samples was dried under a vacuum of 25 inches of mercury for four hours at room temperature. A third set of samples was dried under a vacuum of 25 inches of mercury for four hours at a temperature of 60° C. Microscopy and XRPD indicate that there is minimal, if any, observable difference in particle size or shape between the different drying conditions.

Pharmaceutical compositions of recrystallized compound I can be prepared in conventional dosage unit forms by combining unmilled, recrystallized compound I with a pharmaceutically acceptable carrier according to accepted procedures in an amount sufficient to produce a desired pharmacodynamic activity in a subject, particularly a human. Preferably, the composition contains compound I in an amount selected from about 1 mg to about 500 mg of compound I per dosage unit. The appropriate amount depends on the specific pharmacodynamic activity desired and the condition of the patient. Desirable objects of the compositions and therapeutic methods of the present invention include the treatment of stress, anxiety, premenstrual syndrome (PMS), postnatal depression (PND), and seizures such as those caused by epilepsy. An additional desirable object of the compositions and therapeutic methods is to treat insomnia, and to produce hypnotic activity. Another desirable object of the compositions and therapeutic methods is to induce anesthesia.

The pharmaceutical compositions employed may be, for example, either a solid, liquid, or time release composition (see e.g., “Remington's Pharmaceutical Sciences,” 14th ed., Mack Publishing Company (1970)). Representative solid carriers are lactose, terra alba, sucrose, talc, gelatin, agar, pectin, acacia, magnesium stearate, stearic acid, microcrystalline cellulose, polymer hydrogels and the like. Typical liquid carriers are propylene glycol, glycofurol, aqueous solutions of cyclodextrins, syrup, peanut oil, and olive oil and the like emulsions. Similarly, the carrier or diluent may include any time-delay material known in the art, such as glycerol monostearate or glycerol distearate alone or with wax, microcapsules, microspheres, liposomes, and/or hydrogels.

A wide variety of pharmaceutical forms can be employed. Thus, when using a solid carrier, the preparation can be in oil, tableted, placed in a hard gelatin or enteric-coated capsule in micronized powder or pellet form, or in the form of a troche or lozenge. Compound I may also be administered in the form of a suppository for rectal administration, where compound I can be mixed in material such as cocoa butter and polyethylene glycols or other suitable non-irritating material which is solid at room temperature but liquid at rectal temperature. When using a liquid carrier, the preparation can be in the form of a liquid, such as an ampoule, or as an aqueous or nonaqueous liquid suspension. Liquid dosage forms may also require inclusion of a pharmaceutically acceptable preservative and the like. Parenteral administration, nasal spray, sublingual and buccal administration, and timed release skin patches may also be suitable pharmaceutical forms.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All references cited herein are incorporated by reference in their entireties.

What is claimed is:

1. A pharmaceutical composition, comprising
(a) unmilled, recrystallized 3α-hydroxy-21-(1′-imidazolyl)-3β-methoxymethyl-5α-pregnan-20-one; and
(b) a pharmaceutically acceptable carrier.

2. The pharmaceutical composition of claim 1, wherein said 3α-hydroxy-21-(1′-imidazolyl)-3β-methoxymethyl-5α-pregnan-20-one is recrystallized by cooling a solution of 3α-hydroxy-21-(1′-imidazolyl)-3β-methoxymethyl-5α-pregnan-20-one at a rate of between about 1° C. per hour and about 10° C. per hour.

3. The pharmaceutical composition of claim 2, wherein said rate is about 5° C. per hour.

4. The pharmaceutical composition of claim 2, wherein said solution of 3α-hydroxy-21-(1′-imidazolyl)-3β-methoxymethyl-5α-pregnan-20-one is prepared by
(a) dissolving 3α-hydroxy-21-(1′-imidazolyl)-3β-methoxymethyl-5α-pregnan-20-one in a solvent selected from the group consisting of alkanols (R¹OH), chlorinated hydrocarbons, esters (R¹C(O)OR²), ketones (R¹C(O)R²) and mixtures thereof, wherein R¹ and R² are each independently selected from C₁-C₆ alkyl; and
(b) adding a co-solvent selected from the group consisting of alkanes (CₙH₂ₙ₊₂), ethers (R³OR⁴), glycols and mixtures thereof, wherein n=5 to 12; and R³ and R⁴ are each independently selected from C₁-C₆ alkyl.

5. The pharmaceutical composition of claim 4, wherein said solvent is selected from the group consisting of methanol, ethanol, isopropyl alcohol, dichloromethane, chloroform, ethyl acetate, acetone and mixtures thereof, and said co-solvent is selected from the group consisting of pentane, hexane, heptane, ethyl ether, isopropyl ether, ethylene glycol, propylene glycol and mixtures thereof.

6. The pharmaceutical composition of claim 4 comprising a crystalline methanol solvate of 3α-hydroxy-21-(1′-imidazolyl)-3β-methoxymethyl-5α-pregnan-20-one having substantially the X-ray powder diffraction pattern of FIG. 8.

7. A method for modulating brain excitability in a subject in a manner that alleviates stress, anxiety, insomnia, mood disorders, depression and seizure activity in a mammal, comprising administering to the subject an effective amount of the pharmaceutical composition according to claim 1.

8. The method of claim 7, wherein said effective amount comprises between about 1 mg and about 500 mg of 3α-hydroxy-21-(1′-imidazolyl)-3β-methoxymethyl-5α-pregnan-20-one.

9. A process for recrystallizing 3α-hydroxy-21-(1′-imidazolyl)-3β-methoxymethyl-5α-pregnan-20-one, comprising
(a) dissolving 3α-hydroxy-21-(1′-imidazolyl)-3β-methoxymethyl-5α-pregnan-20-one in a solvent selected from the group consisting of alkanols (R¹OH), chlorinated hydrocarbons, esters (R¹C(O)OR²), ketones (R¹C(O)R²) and mixtures thereof, wherein R¹ and R² are each independently selected from C₁-C₆ alkyl;
(b) adding a co-solvent selected from the group consisting of alkanes (CₙH₂ₙ₊₂), ethers (R³OR⁴), glycols and mixtures thereof to form a solution, wherein n=5-12; and R³ and R⁴ are each independently selected from C₁-C₆ alkyl;
(c) cooling said solution at a rate of between about 1° C. per hour and about 10° C. per hour; and
(d) collecting the resulting crystals,
wherein said resulting crystals have substantially the crystal shape and size of FIG. 11.

10. A crystalline form of 3α-hydroxy-21-(1′-imidazolyl)-3β-methoxymethyl-5α-pregnan-20-one having substantially the crystal shape and size of the majority of crystals presented in FIG. 11.

11. A preparation comprising crystals of 3α-hydroxy-21-(1′-imidazolyl)-3β-methoxymethyl-5α-pregnan-20-one having an average crystal size of less than about 100 μm.

12. The preparation of claim 11, wherein the average crystal size is less than about 50 μm.

13. The preparation of claim 11, wherein the average crystal size is less than about 25 μm.
